(19) United States
(12) Patent Application Publication
(10) Pub. No.: US 2010/0160870 A1
(43) Pub. Date: Jun. 24, 2010

(54) Two Piece Housing For Drug Loading

(75) Inventors: Don A. Clements, Cleburne, TX (US); Julie A. Clifford, Arlington, TX (US); Michell R. Dupre, Mansfield, TX (US); Mark T. DuPriest, Hudson Oaks, TX (US); Michael J. Piazza, Crowley, TX (US); Pardeep K. Sethi, Forth Worth, TX (US); Roger Vanderlaan, Arlington, TX (US)

Correspondence Address: ALCON, IP LEGAL, TB4-8, 6201 SOUTH FREEWAY, FORT WORTH, TX, 76134, US

(73) Assignee: Alcon Research, LTD., Fort Worth, TX (US)

(21) Appl. No.: 12/636,857

(22) Filed: Dec. 14, 2009

Related U.S. Application Data
(60) Provisional application No. 61/138,663, filed on Dec. 18, 2008.

(51) Int. Cl. A61M 5/31

(52) U.S. Cl. 604/218

(57) ABSTRACT

A drug loading apparatus has a dispensing chamber housing formed by a top housing part and a bottom housing part. The top housing part is separable from the bottom housing part. A plunger is located in the bottom housing part such that the plunger forms a first fluid seal with an inner wall of the bottom housing part. When the top housing part is coupled to the bottom housing part, a second fluid seal is formed.

The present invention relates to loading a drug into an injection device and more particularly to an apparatus and system for loading a drug into a two piece hub. This application claims the benefit of U.S. Provisional Patent Application No. 61/138,663 filed Dec. 18, 2008.

### BACKGROUND OF THE INVENTION

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

These, and other diseases, can be treated by injecting a drug into the eye. Such injections are typically done manually using a conventional syringe and needle. FIG. 1 is a perspective view of a prior art syringe used to inject drugs into the eye. In FIG. 1, the syringe includes a needle 105, a luer hub 110, a chamber 115, a plunger 120, a plunger shaft 125, and a thumb rest 130. As is commonly known, the drug to be injected is located in chamber 115. Pushing on the thumb rest 130 causes the plunger 120 to expel the drug through needle 105.

In using such a syringe, the surgeon is required to pierce the eye tissue with the needle, hold the syringe steady, and actuate the syringe plunger (with or without the help of a nurse) to inject the fluid into the eye. Fluid flow rates are uncontrolled. The volume injected is typically not controlled in an accurate manner because reading the vernier is subject to parallax error. Tissue damage may occur due to an “unsteady” injection.

An effort has been made to control the delivery of small amounts of liquids. A commercially available fluid dispenser is the ULTRA™ positive displacement dispenser available from EFD Inc. of Providence, R.I. The ULTRA dispenser is typically used in the dispensing of small volumes of industrial adhesives. It utilizes a conventional syringe and a custom dispensing tip. The syringe plunger is actuated using an electrical stepper motor and an actuating fluid. Parker Hannifin Corporation of Cleveland, Ohio distributes a small volume liquid dispenser for drug discovery applications made by Aurora Instruments LLC of San Diego, Calif. The Parker/Aurora dispenser utilizes a piezo-electric dispensing mechanism. Ypsomed, Inc. of Switzerland produces a line of injection pens and automated injectors primarily for the self-injection of insulin or hormones by a patient. This product line includes simple disposable pens and electronically-controlled motorized injectors.

U.S. Pat. No. 6,290,690 discloses an ophthalmic system for injecting a viscous fluid (e.g. silicone oil) into the eye while simultaneously aspirating a second viscous fluid (e.g. perflourocarbon liquid) from the eye in a fluid/fluid exchange during surgery to repair a retinal detachment or tear. The system includes a conventional syringe with a plunger. One end of the syringe is fluidly coupled to a source of pneumatic pressure that provides a constant pneumatic pressure to actuate the plunger. The other end of the syringe is fluidly coupled to an infusion cannula via tubing to deliver the viscous fluid to be injected.

When a portable hand piece is used to inject a drug into the eye, it is important to provide a proper drug dosage. In one case, a phase transition compound or reverse gelation compound contains the drug. At room temperature, these compounds are in a solid state and have the consistency of wax. Because of their consistency, dosing an injector with these compounds can be difficult. The compounds can be brought to a more liquid state and drawn into the injector. However, this is a time consuming process that may not provide proper dosage. It would be desirable to have a system for accurately and quickly loading such a drug mixture into an injection device.

### SUMMARY OF THE INVENTION

In one embodiment consistent with the principles of the present invention, the present invention is a drug loading apparatus with a dispensing chamber housing formed by a top housing part and a bottom housing part. The top housing part is separable from the bottom housing part. A plunger is located in the bottom housing part such that the plunger forms a first fluid seal with an inner wall of the bottom housing part. When the top housing part is coupled to the bottom housing part, a second fluid seal is formed.

In another embodiment consistent with the principles of the present invention, the present invention is a method of loading an injection device including placing a plunger in a bottom housing part of a dispensing chamber housing to form a dispensing chamber bounded by a top face of the plunger and an inner wall of the bottom housing part; dispensing a substance into the dispensing chamber; and sealing a top housing part to the bottom housing part.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying figures, which are incorporated in and constitute a part of this specification, illustrate several embodiments of the invention and together with the description, serve to explain the principles of the invention.

FIG. 1 is a perspective view of a prior art syringe.

FIG. 2 is a cross section view of a disposable tip segment and a limited reuse assembly according to the principles of the present invention.

FIG. 3A is a cross section view of an unassembled two piece housing and plunger according to the principles of the present invention.

FIG. 3B is a cross section view of an assembled two piece housing and plunger according to the principles of the present invention.

FIG. 4A is a cross section view of the loading process for a two piece housing and plunger according to the principles of the present invention.

FIG. 4B is a cross section view of the loading process for a two piece housing and plunger according to the principles of the present invention.

FIG. 4C is a cross section view of the loading process for a two piece housing and plunger according to the principles of the present invention.

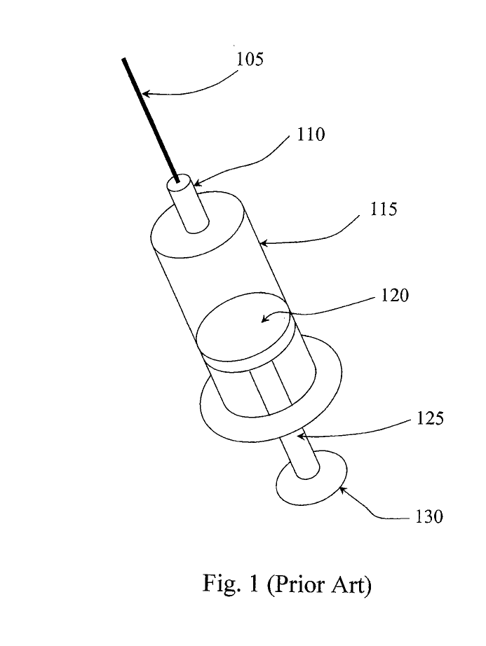

### DETAILED DESCRIPTION OF THE PREFERRED EMBODIMENTS

Reference is now made in detail to the exemplary embodiments of the invention, examples of which are illustrated in the accompanying figures. Wherever possible, the same reference numbers are used throughout the figures to refer to the same or like parts.

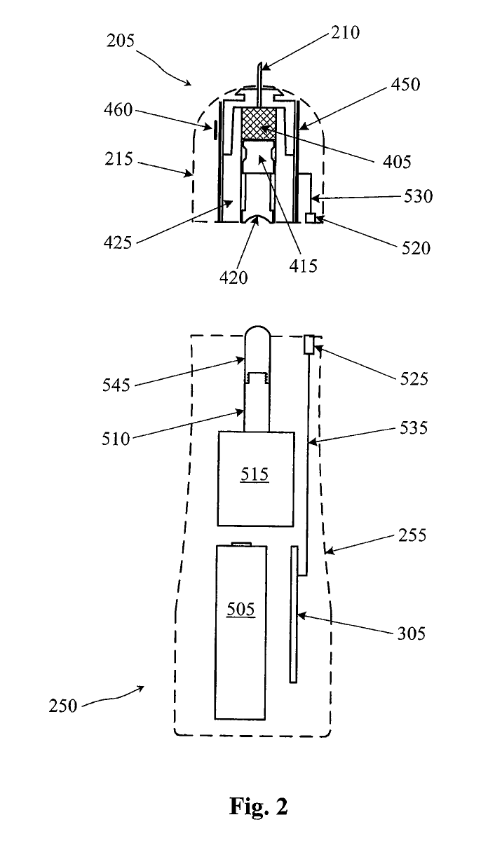

FIG. 2 is a cross section view of a disposable tip segment and a limited reuse assembly according to an embodiment of the present invention. FIG. 2 shows how tip segment 205 interfaces with limited reuse assembly 250. In the embodiment of FIG. 2, tip segment 205 includes plunger interface 420, plunger 415, dispensing chamber housing 425, tip segment housing 215, temperature control device 450, thermal sensor 460, needle 210, dispensing chamber 405, interface 530, and tip interface connector 520. Limited reuse assembly 250 includes mechanical linkage 545, actuator shaft 510, actuator 515, power source 505, controller 305, limited reuse assembly housing 255, interface 535, and limited reuse assembly interface connector 525.

In tip segment 205, plunger interface 420 is located on one end of plunger 415. The other end of plunger 415 forms one end of dispensing chamber 405. Plunger 415 is adapted to slide within dispensing chamber 405. An outer surface of plunger 415 is fluidly sealed to the inner surface of dispensing chamber housing 425. Dispensing chamber housing 425 surrounds the dispensing chamber 405. Typically, dispensing chamber housing 425 has a cylindrical shape. As such, dispensing chamber 405 also has a cylindrical shape.

Needle 210 is fluidly coupled to dispensing chamber 405. In such a case, a substance contained in dispensing chamber 405 can pass through needle 210 and into an eye. Temperature control device 450 at least partially surrounds dispensing chamber housing 425. In this case, temperature control device 450 is adapted to heat and/or cool dispensing chamber housing 425 and any substance contained in dispensing chamber 405. Interface 530 connects temperature control device 450 and thermal sensor 460 with tip interface connector 520.

The components of tip segment 205, including dispensing chamber housing 425, temperature control device 450, and plunger 415 are at least partially enclosed by tip segment housing 215. In one embodiment consistent with the principles of the present invention, plunger 415 is sealed to the interior surface of dispensing chamber housing 425. This seal prevents contamination of any substance contained in dispensing chamber 405. For medical purposes, such a seal is desirable. This seal can be located at any point on plunger 415 or dispensing chamber housing 425.

In limited reuse assembly 250, power source 505 provides power to actuator 515. An interface (not shown) between power source 505 and actuator 515 serves as a conduit for providing power to actuator 515. Actuator 515 is connected to actuator shaft 510. When actuator 515 is a stepper motor, actuator shaft 510 is integral with actuator 515. Mechanical linkage interface 545 is connected to actuator shaft 510. In this configuration, as actuator 515 moves actuator shaft 510 upward toward needle 210 mechanical linkage interface 545 also moves upward toward needle 210.

Controller 305 is connected via interface 535 to limited reuse assembly interface connecter 525. Limited reuse assembly interface connecter 525 is located on a top surface of limited reuse assembly housing 255 adjacent to mechanical linkage interface 545. In this manner, both limited reuse assembly interface connector 525 and mechanical linkage interface 545 are adapted to be connected with tip interface connector 520 and plunger interface 420 respectively.

Controller 305 and actuator 515 are connected by an interface (not shown). This interface (not shown) allows controller 305 to control the operation of actuator 515. In addition, an interface (not shown) between power source 505 and controller 305 allows controller 305 to control operation of power source of 310. In such a case, controller 305 may control the charging and the discharging of power source 505 when power source 505 is a rechargeable battery.

Controller 305 is typically an integrated circuit with power, input, and output pins capable of performing logic functions. In various embodiments, controller 305 is a targeted device controller. In such a case, controller 305 performs specific control functions targeted to a specific device or component, such as a temperature control device or a power supply. For example, a temperature control device controller has the basic functionality to control a temperature control device. In other embodiments, controller 305 is a microprocessor. In such a case, controller 305 is programmable so that it can function to control more than one component of the device. In other cases, controller 305 is not a programmable microprocessor, but instead is a special purpose controller configured to control different components that perform different functions. While depicted as one component, controller 305 may be made of many different components or integrated circuits.

Tip segment 205 is adapted to mate with or attach to limited reuse assembly 250 as previously described. In the embodiment of FIG. 5, plunger interface 420 located on a bottom surface of plunger 415 is adapted to mate with mechanical linkage interface 545 located near a top surface of limited reuse assembly housing 255. In addition, tip interface connector 520 is adapted to connect with limited reuse assembly interface connector 525. When tip segment 205 is connected to limited reuse assembly 250 in this manner, actuator 515 and actuator shaft 510 are adapted to drive plunger 415 upward toward needle 210. In addition, an interface is formed between controller 305 and temperature control device 450. A signal can pass from controller 305 to temperature control device 450 through interface 535, limited reuse assembly interface connector 525, tip interface connector 520, and interface 530.

In operation, when tip segment 205 is connected to limited reuse assembly 250, controller 305 controls the operation of actuator 515. Actuator 515 is actuated and actuator shaft 510 is moved upward toward needle 210. In turn, mechanical linkage interface 545, which is mated with plunger interface 420, moves plunger 415 upward toward needle 210. A substance located in dispensing chamber 405 is then expelled through needle 210.

In addition, controller 305 controls the operation of temperature control device 450. Temperature control device 450 is adapted to heat and/or cool dispensing chamber housing 425. Since dispensing chamber housing 425 is at least partially thermally conductive, heating or cooling dispensing chamber housing 425 heats or cools a substance located in dispensing chamber 405. Temperature information can be transferred from thermal sensor 460 to controller 305 via any of a number of different interface configurations. This temperature information can be used to control the operation of temperature control device 450. When temperature control device 450 is a heater, controller 305 controls the amount of current that is sent to temperature control device 450. The more current sent to temperature control device 450, the hotter it gets. In such a manner, controller 305 can use a feed back loop utilizing information from thermal sensor 460 to control the operation of temperature control device 450. Any suitable type of control algorithm, such as a proportional integral derivative (PID) algorithm, can be used to control the operation of temperature control device 450.

In various embodiments of the present invention, temperature control device 450 heats a phase transition compound that is located in dispensing chamber 405. This phase transition compound carries a drug that is to be injected into the eye. A phase transition compound is in a solid or semi-solid state at lower temperatures and in a more liquid state at higher temperatures. Such a substance can be heated by temperature control device 450 to a more liquid state and injected into the eye where it forms a bolus that erodes over time. Likewise, a reverse gelation compound may be used. A reverse gelation compound is in a solid or semi-solid state at higher temperatures and in a more liquid state at lower temperatures. Such a compound can be cooled by temperature control device 450 to a more liquid state and injected into the eye where it forms a bolus that erodes over time. As such, temperature control device 450 may be a device that heats a substance in dispensing chamber 405 or a device that cools a substance in dispensing chamber 405 (or a combination of both). After being delivered into the eye, a phase transition compound or reverse gelation compound erodes over time providing a quantity of drug over an extended period of time. Using a phase transition compound or reverse gelation compound provides better drug dosage with fewer injections.

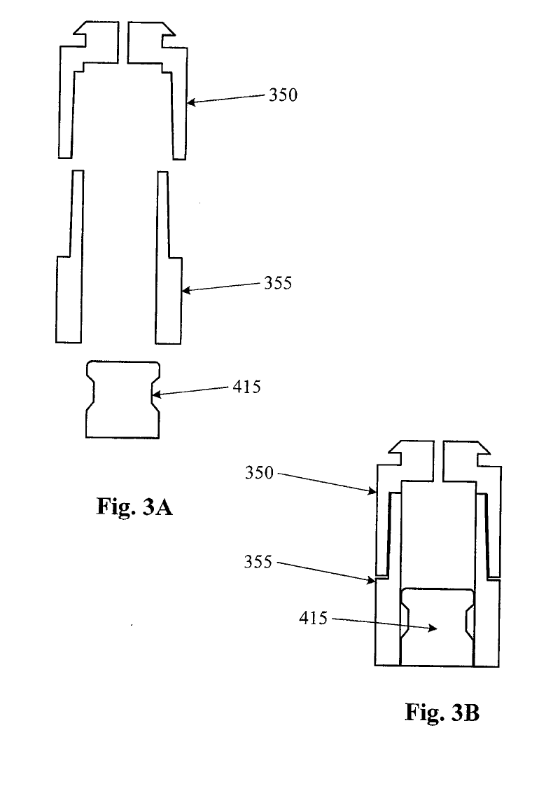

FIG. 3A is a cross section view of an unassembled two piece hub and plunger according to the principles of the present invention. FIG. 3B is a cross section view of an assembled two piece hub and plunger according to the principles of the present invention. In FIG. 3A, the dispensing chamber housing 425 is made of two pieces—top housing part 350 and bottom housing part 355. Plunger 415 is also depicted. Top housing part 350 has a through hole in its top surface to accept a needle for dispensing a substance. The inner wall of top housing part 350 is designed to engage the outer wall of bottom housing part 355 to form a press fit seal when pressed together as shown in FIG. 3B. Plunger 415 fits into bottom housing part 355 as shown to form a fluid seal. When assembled, the two part housing 350, 355 forms a single dispensing chamber housing 425 suitable for holding a substance that is to be delivered into the eye.

While depicted as a press fit connection, top housing part 350 and bottom housing part 355 may be connected together using any mechanical means. For example, top housing part 350 and bottom housing part 355 may be connected via a threaded connection. Other structures (such as ridges grooves, barbs or the like) may be present on the inner wall of top housing part 350 and/or the outer wall of bottom housing part 355. Further, the inner wall of top housing part 350 and/or the outer wall of bottom housing part 355 may be sloped to facilitate a fluid tight seal. Top housing part 350 and bottom housing part 355 may be made of any suitable material (preferably thermally conductive).

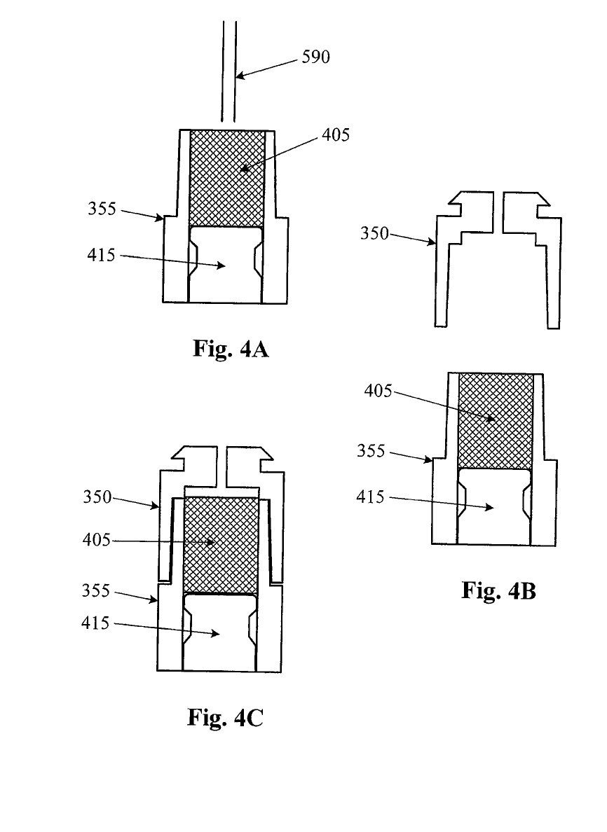

FIGS. 4A-4C are a cross section views of the loading process for a two piece hub and plunger according to the principles of the present invention. In FIG. 4A, plunger 415 is inserted into bottom housing part 355. Since plunger 415 forms a fluid tight seal with the inner wall of bottom housing part 355, a dispensing chamber is located above plunger 415. A substance 405 is injected into this dispensing chamber via cannula 590. When substance 405 is a phase transition compound/drug mixture, the substance must be heated so that it can flow into the dispensing chamber. In this manner, cannula 590 and/or bottom housing part 355 may be heated to allow the substance 405 to flow. This allows for the precise dosing of a viscous and difficult to dose substance. While shown as full, any dosage of substance can be loaded into the dispensing chamber. Further, this method allows for little or no air to be entrapped between the plunger 415 and the substance 405 and between the inner wall of bottom housing part 355 and the substance 405.

After the substance 405 is loaded into the dispensing chamber, top housing part 350 is fitted onto bottom housing part 355 as previously described. An air gap located above substance 405 can be controlled by controlling the interior dimensions of top housing part 350 and the fill level of the substance.

This drug loading process has significant advantages over traditional processes. This process is capable of being automated so that a number of injection devices can be filled. In addition, dosing can be much more precise as can placement of the dosage in the device. Loading a drug mixture in a pharmaceutical facility in such a manner also increases the safety of the device—it keeps the mixture in a controlled environment, precisely doses the mixture, and allows for other inspections and quality controls.

From the above, it may be appreciated that the present invention provides an improved system for preparing drug dosage. The present invention provides an apparatus that is designed to reliably make pellets of a consistent quality. This apparatus is configured to form pellets from a drug/compound mixture that is solid at room temperature but liquid at other temperatures. The finished pellets are of the proper size to produce a reliable dosage when injected into the eye.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An apparatus comprising:
a dispensing chamber housing formed by a top housing part and a bottom housing part, the top housing part separable from the bottom housing part; and
a plunger located in the bottom housing part such that the plunger forms a first fluid seal with an inner wall of the bottom housing part;
wherein when the top housing part is coupled to the bottom housing part, a second fluid seal is formed.

2. The apparatus of claim 1 further comprising:
a drug loading apparatus configured to inject a mixture into the bottom housing part on top of the plunger.

3. The apparatus of claim 2 wherein the drug loading apparatus is heated.

4. The apparatus of claim 1 further comprising:
a temperature control device at least partially surrounding the bottom housing part.

5. The apparatus of claim 1 wherein the top housing part is coupled to the bottom housing part via a press fit connection.

6. A method of loading an injection device comprising:
placing a plunger in a bottom housing part of a dispensing chamber housing to form a dispensing chamber bounded by a top face of the plunger and an inner wall of the bottom housing part;
dispensing a substance into the dispensing chamber; and
sealing a top housing part to the bottom housing part.

7. The method of claim 6 further comprising:
heating the substance.

8. The method of claim 12 further comprising:
heating the bottom housing part.
